(19) United States
(12) Patent Application Publication
(10) Pub. No.: US 2026/0338696 A1
(43) Pub. Date: Oct. 29, 2026

(54) RADIATION EMISSION COMPONENT FOR THE TEMPERATURE-COMPENSATED OPTICAL DETECTION OF AN OXYGEN CONTENT OF A FLUID

(71) Applicant: Hamilton Medical AG, Bonaduz (CH)

(72) Inventors: Eva-Maria Grether, Konstanz (DE); Philipp Baur, Chur (CH)

(21) Appl. No.: 19/118,300

(22) PCT Filed: Oct. 18, 2023
(86) PCT No.: PCT/EP2023/078946

§ 371 Date: Apr. 3, 2025

(30) Foreign Application Priority Data
Oct. 19, 2022 (DE) 10 2022 127 603.1

(51) Int. Cl. G01N 21/77; G01N 21/03; G01N 21/05

(52) U.S. Cl. CPC G01N 21/77; G01N 21/05; G01N 2021/0389; G01N 2021/7763; G01N 2021/7786; G01N 2201/1211

(57) ABSTRACT

A radiation emission component for the temperature-compensated optical detection of an oxygen content of a fluid, wherein the radiation emission component includes a contact surface wettable by the fluid, and a coupling surface different from the contact surface, wherein the radiation emission component has a luminophore-containing region, wherein the luminophore is reachable by oxygen in a first portion of the contact surface for the quenching thereof, wherein the radiation emission component has a temperature detection region arranged offset along the contact surface relative to the luminophore-containing region, and wherein the radiation emission component has, in the temperature detection region, a main body having a cutout extending toward the contact surface, wherein the cutout is covered by a base having a base layer body, wherein a surface of the base layer body facing away from the cutout forms a second portion of the contact surface; wherein the main body and the base layer body each to include plastics material, wherein the main body and the base layer body are integrally connected to one another by way of their respective plastics materials.

This Application claims priority in PCT application PCT/EP 2023/078946 filed Oct. 18, 2023, which claims priority in German Patent Application DE 10 2022 127 603.1 filed on Oct. 19, 2022, which are incorporated by reference herein.

The present invention concerns a radiation emission component for the temperature-compensated optical detection of an oxygen content of a fluid, where the radiation emission component comprises a contact surface wettable by the fluid and a coupling surface different from the contact surface for coupling with a radiation-sensitive sensor arrangement.

The radiation emission component exhibits a luminophore-containing region. Typically, a luminophore in the luminophore-containing region can be excited through irradiation with a first electromagnetic radiation of a first wavelength to radiate a second electromagnetic radiation of a second wavelength different from the first wavelength. The excited radiation behavior of the luminophore in the luminophore-containing region depends on a partial oxygen pressure in a fluid contacting the contact surface. The luminophore can be reached by oxygen in a first section of the contact surface. Its emission of excited radiation can be quenched through contact of the luminophore with oxygen molecules in a manner which is known per se.

The radiation emission component moreover exhibits an infrared radiation-emitting temperature detection region. The temperature detection region is arranged offset relative to the luminophore-containing region in a reference direction along the contact surface. The radiation emission component exhibits in the section in which the temperature detection region is arranged a main body with a recess extending towards the contact surface. The recess is covered by a base comprising a base layer body. A surface of the base layer body facing away from the recess forms a second section of the contact surface different from the first section.

### BACKGROUND OF THE INVENTION

Such a radiation emission component is known as a layer body from WO 2018/166847 A1. In the known radiation emission component, the base layer body is preferably made from a thermally well-conducting metal, in particular from aluminum. The known base layer body of metal foil is glued onto the side of the main body which during operation faces towards the measurement fluid on its front face, in such a way that it covers a recess penetrating through the main body. The metal foil, that is, the base layer body, can be coated with a black varnish on its side facing the recess in order to achieve a higher emissivity in the relevant wavelength range than would be the case with the blank metal surface.

Such radiation emission components are preferably arranged in ventilation lines of ventilation devices for artificial ventilation of patients for the measurement of the oxygen content of respiratory gas, for example in a measuring cuvette through which respiratory gas flows, which is couplable with a radiation-sensitive sensor arrangement for radiation exchange. The measurement fluid is then a measurement gas, namely the respiratory gas.

Beyond the partial oxygen pressure of the respiratory gas, the quenching of the luminophore, known per se, used by the radiation emission component for detecting the oxygen content in the respiratory gas of a ventilated patient depends on the temperature of the luminophore. In order to be able to evaluate in a compensating manner the second electromagnetic radiation radiated from the luminophore-containing region in terms of the unavoidable temperature impact and infer the most correct possible oxygen content in the respiratory gas, the temperature detection region whose emitted infrared radiation is a measure of its temperature is arranged at the specified spatial offset from the luminophore-containing region. Due to the recess and the comparatively thin configuration of the base of the recess, formed in the state of the art through the if desired coated metal foil, the temperature of the base or to be precise the temperature of the free surface of the base facing towards the recess is approximately equal to the temperature of the luminophore in the luminophore-containing region. Due to the base, which covers the recess, the recess is configured in the manner of a blind hole.

Consequently, for temperature compensation of the detected quenching effect, a temperature spatially distant from the luminophore which sufficiently accurately corresponds to the temperature of the luminophore can be detected, such that the two radiation detections, one time in the luminophore-containing region and a further time in the temperature detection region, do not mutually disrupt each other.

The statements made above regarding the field of application and the mode of operation of the radiation emission component and its regions: luminophore-containing region and temperature detection region, apply to the state of the art radiation emission component just as they apply to the here presented radiation emission component according to the invention.

However, the fabrication of the known radiation emission component is costly, since the metal foil, where applicable after coating, has to be glued onto the main body of the temperature detection region which usually is made from a synthetic material. This gluing process is not only cumbersome in its course through glue application and positioning relative to one another of the components which are to be glued, through disruption-free curing of the glue etc, but also requires the use of specially matched glues, since in particular aluminum foils cannot be readily glued. Metal foils either require pretreatment, for instance through the application of a primer as a bonding agent layer, or a special glue has to be chosen which is able to achieve durable gluing of a metal foil with the main body. Detachment of the metal foil from the main body on the contact side is unacceptable precisely in ventilation applications, since then the detached metal foil can be swept along through the respiratory gas stream towards the patient.

### SUMMARY OF THE INVENTION

It is, therefore, the task of the present invention to provide a radiation emission component working without loss of accuracy, which with can be operate with great reliability and which nevertheless is simple to fabricate.

This task is solved according to the present invention in a radiation emission component of the type mentioned in the beginning by having the main body and the base layer body each comprise a synthetic material, where the main body and the base layer body are connected integrally with one another by means of their respective synthetic materials. The integral connection is recognizably an especially secure connection. To this end it is necessary to design each of the base layer body and the main body with synthetic material, in order to be able to provide the integral material connection through the two synthetic materials.

Recognizably, synthetic material normally has lower heat conductance than the metal foil known from the state of the art. This drawback of the base, however, can be compensated for through a sufficiently thin design of the base between its contact surface which is wettable by measurement fluid and its opposite free surface which radiates infrared radiation towards the recess.

The contact surface of the radiation emission component is wettable by fluid, which in principle indicates that the contact surface of the radiation emission component is an exposed surface of same. This ensures the wettability by the measurement fluid to be detected.

The coupling surface of the radiation emission component is likewise an exposed surface. It is spatially arranged and/or configured as the case may be at a different location than the contact surface and therefore differs from the contact surface. The coupling surface, as an exposed surface of the main body, preferably lies opposite the contact surface along a path and/or axis of the depth extension of the recess. In this case, the recess extends in the manner of a blind hole starting from a normally free surface of the main body lying opposite the contact surface towards the contact surface. The coupling surface serves for coupling the radiation-sensitive sensor arrangement. The first electromagnetic radiation can preferably be introduced through the coupling surface into the main body for radiation excitation of the luminophore in the luminophore-containing region. Likewise preferably, the second electromagnetic radiation radiated by the luminophore is emitted through the coupling surface out of the main body. In addition, infrared radiation emitted from the coupling surface is detected by an infrared sensor of the radiation-sensitive sensor arrangement.

In principle it is possible that the infrared radiation emitted from the base of the recess in the temperature detection region is radiated in a different direction than the second electromagnetic radiation in the luminophore-containing region, that is, that the coupling surface exhibits differently oriented regions in the luminophore-containing region and in the temperature detection region. Preferably, the coupling surface of the radiation emission component over the luminophore-containing region and the temperature detection region is oriented uniformly, in order on the one hand to configure the coupling surface with the smallest possible area and/or installation space requirement and in order on the other hand to facilitate coupling of the radiation-sensitive sensor arrangement.

According to a first possible embodiment of the radiation emission component, it can be provided that the synthetic material of the main body and the synthetic material of the base layer body are fused with one another regionally. This makes possible separate fabrication of the main body on the one hand and of the base layer body on the other with sufficiently compatible synthetic materials, such that these can be fused directly with one another through a welding method, such as for example ultrasound welding or mirror welding, to name only two possible examples, without the mediation of glue. This first possible embodiment permits great structural freedom in the design in particular of the base layer body, but requires with the joining process for the fusing of main body and base layer body an additional working step in the fabrication of the radiation emission component.

According to a second possible embodiment, the main body and the base layer body can be made from the same synthetic material integrally-monolithically. Although machining fabrication of the temperature detection region through machining material removal in the region of the recess while leaving undisturbed the base made of solid material is conceivable in principle, such machining fabrication is not preferred due to the long fabrication duration and the necessary time-consuming machining processes in preparing for the machining material removal. More preferable is an integral fabrication of main body, recess, and base through a primary forming method, such as for instance molding or especially preferably injection molding. According to current findings, the base layer body with a thickness in the region of between 200 μm to 300 μm thickness fabricated integrally with the main body can be fabricated without difficulties. Should smaller thicknesses of the base layer body be required, then for thicknesses in the region of 100 μm to 200 μm an injection-compression molding method can be used, in which initially a blank which is close to the final form is injection-molded and still in the injection-molded form, with a slider used as a compression stamp, the base region of the recess and thereby the base layer body is postformed in a second shaping process. The layer thicknesses achievable through injection molding alone or through injection-compression molding depend on the respective process control and on the respective synthetic material used. Certainly there exists in the thickness range from 80 to 120 μm, depending on the synthetic material that is to undergo primary forming, a certain choice in fabricating the base region through one or the other method integrally with the main body. This integral-monolithic fabrication of main body and base layer body achieves the most durable and firmest connection between the aforementioned bodies.

In order to make sure that the base layer body can be connected integrally with the main body in one manner or the other, the base layer body is preferably formed to 100% out of synthetic material. The synthetic material can be a synthetic filled with a filler material, for instance particles and/or fibers, in order to increase its strength, such as tensile strength and bending strength. The filler material can, but does not have to, consist of a synthetic or comprise a synthetic. The synthetic material can alternatively be an unfilled synthetic, which in terms of the spatially most homogeneous possible material properties of the temperature detection section is preferable. Furthermore, an optically transparent base layer body is achievable by means of an unfilled synthetic, which normally is not possible with a filled synthetic. For simple fabrication of the integral connection, the synthetic is preferably a thermoplastic synthetic, regardless of whether it is used as a matrix synthetic of a filled synthetic material or as an unfilled synthetic.

In particular when the base layer body is fabricated separately from the main body and is joined with it integrally through a welding process, it can be designed as a synthetic foil. The synthetic foil can be formed out of several layers, in particular synthetic material layers. Thus the synthetic foil can exhibit a temperature information layer with an especially advantageous low transmittivity in the infrared wavelength region relevant for the planned infrared temperature measurement. Furthermore, the temperature information layer can exhibit an external connecting layer on its side facing towards the main body and/or towards the recess, as the case may be, which can be connected especially well with the synthetic of the main body in a fusing manner, in particular through welding or sealing. The connecting layer can be thinner than the temperature information layer. Since, for the simplest possible configuration of the temperature detection region, the base layer body forms the aforementioned second section of the contact surface and thereby an exposed surface facing away from the recess in the main body, a surface of the temperature information layer can form the second section of the contact surface.

The first and the second section of the contact surface, in order to avoid reciprocal undesirable effects of the respective relevant radiations, are arranged offset relative to one another in a reference direction along the contact surface, for instance in the longitudinal direction of the radiation emission component. Optionally, the first and the second section of the contact surface can additionally or alternatively be arranged offset relative to one another in the reference direction parallel to the contact surface.

In order to achieve the simplest possible manufacturability and fabrication of the temperature detection region, the base layer body preferably comprises only one type of synthetic. This type of synthetic can for example be styrene, polyester, polyolefin etc, to name only a few examples. The main body also preferably comprises only one type of synthetic. Normally, different synthetics of the same type are compatible with one another and can through fusing be connected integrally with one another.

Although different synthetics of one and the same type of synthetic, such as for example polyethylene and polypropylene as polyolefins, are compatible with one another, the base layer body, and preferably also the main body, especially preferably comprises only one synthetic. This synthetic is, in particular, preferably the same synthetic in the integral-monolithic configuration for both components out of main body and base layer body. This synthetic is preferably the synthetic methyl methacrylate acrylonitril butadiene styrene (MABS) which is already proven in applications for medical devices. Especially preferably, the base layer body is a one-layer body with only a single layer, as can be obtained for instance in injection molding or injection-compression molding. A separate base layer body too, configured for instance as a foil, can be a one-layer body. Thus to the simplification through the use of preferably only a single synthetic there is added the further simplification through configuring the base layer body in only one single layer. This, however, does not rule out that subsequently one or several coatings are applied onto the base layer body, for instance in order to match the emission behavior and/or the absorption behavior of the base to the planned application of the detection of the emitted infrared radiation.

In the nomenclature of the present application, a coating applied viscously subsequently onto a previously fabricated base layer body and then cured and/or dried is indeed part of the base, but not part of the base layer body.

In order on the one hand to be able to ensure rapid matching of the temperature of the base layer body to the temperature of the luminophore in the luminophore-containing region under a varying temperature of the measurement fluid wetting the contact surface and in order on the other hand to be able to guarantee a sufficiently stable configuration of the temperature detection region, the base layer body is preferably configured as thinner than the main body surrounding the recess. The base layer body and/or the base of the temperature detection region, as the case may be, can exhibit a thickness in the range of 50 μm to 300 μm. In order to provide sufficient rigidity and strength, the main body is at least 500 μm, preferably at least 1 mm, especially preferably several millimeters thick.

The thickness of the radiation emission component in the temperature detection region is preferably measured along the direction of the depth extension of the recess.

The section of the coupling surface through which the first electromagnetic radiation is introduced into the main body and/or the second electromagnetic radiation is emitted out of it, preferably forms a first section of the coupling surface. At this first section of the coupling surface, preferably only the first electromagnetic radiation is introduced and the second electromagnetic radiation is emitted, but no infrared radiation detected.

The exposed surface of the base which faces towards the recess and is opposite the second section of the contact surface forms a second section of the coupling surface. In order to avoid mutual disruption in the detection of infrared radiation on the one hand and in the emission of the first electromagnetic radiation and the detection of the second electromagnetic radiation on the other, the second section of the coupling surface is arranged offset relative to the first section of the coupling surface in a direction along the coupling surface, in particular parallel to the coupling surface. At the second section of the coupling surface, preferably only the infrared radiation radiated from the second section is detected, but no radiation to or from the luminophore-containing region.

Because of the configuration of the recess, the second section of the coupling surface is arranged offset relative to the first section of the coupling surface in the direction of the depth extension of the recess. As already explained above, the direction of the depth extension of the recess corresponds to the thickness direction of the main body. The coupling surface and the contact surface are preferably parallel to one another. The first sections of the contact and of the coupling surface preferably lie opposite one another in the thickness direction of the main body. Additionally or alternatively, the second sections of the contact and of the coupling surface lie opposite one another in the thickness direction of the main body, in particular of the base of the recess. Further preferably, the second section of the coupling surface is also arranged offset in the reference direction relative to the first section of the coupling surface.

A synthetic material of the base layer body advantageous for the temperature detection through infrared radiation emission preferably exhibits for infrared radiation in a wavelength region from 5500 nm to 14000 nm a transmittivity not exceeding 20%. Preferably, the entire thickness region of the base layer body between its opposite outer surfaces is made from the synthetic material with the low transmittivity of 20% or less.

Even more preferably, the synthetic material of the base layer body exhibits for infrared radiation in the wavelength region a transmittivity not exceeding 5%. The aforementioned synthetic MABS exhibits in the wavelength region the advantageously low transmittivity not exceeding 5%. Thereby it can be ensured that infrared radiation emitted for instance from the measurement gas and/or measurement fluid, as the case may be, beyond the contact surface does not reach an infrared sensor, but rather infrared radiation actually emitted from the base layer body. Thus faulty measurements can be avoided in which the temperature of the measurement fluid replaces the temperature of the base layer body and/or of the second section of the coupling surface, as the case may be. Due to the advantageously small transmittivity not exceeding 5%, the synthetic material of the base layer body can directly form the surface of the base facing towards the recess. Consequently, the surface of an injection-molded or injection-compressed base layer body or a foil surface of a base layer body configured as a foil can form the surface facing towards the recess and/or the second section of the coupling surface, respectively. An additional coating for matching the radiation behavior of the base to the metrological constraints is advantageously not required in this case.

If, however, the transmittivity of the synthetic of the base layer body in the relevant wavelength region is too high, the base layer body can have a coating on its surface facing away from the contact surface and/or on its surface facing towards the contact surface or forming a section of the contact surface, respectively. If a coating is required, its arrangement on the surface facing away from the contact surface is preferable, since the latter faces directly towards an infrared sensor arranged on the side of the coupling surface and can preferably only lie opposite it under interposition of an air gap. This coating is preferably applied subsequently onto the base layer body. The coating is preferably chosen in such a way that the base exhibits in the direction towards the recess in the wavelength region from 5500 nm to 14000 nm an absorptance of at least 95% and/or an emissivity of at least 95%. The coating is preferably made from a material which exhibits an absorptance of at least 95% and/or an emissivity of at least 95%. Such a coating is available for example as a deep black coating from ACM COATINGS GmbH, Rudelsburgpromenade 20c, in 06628 Naumburg—Bad Kösen (DE).

In order to avoid disruptions through interfering radiation, preferably the temperature detection region, in particular the base, especially preferably the base layer body, is free from luminophore.

Just like the temperature detection region, the radiation emission component can exhibit a luminophore main body. For doping the luminophore-containing region of the radiation emission component, the luminophore main body can carry an oxygen-permeable foil. A first surface of the foil preferably forms the first section of the contact surface of the radiation emission component. Likewise preferably, the luminophore is arranged between a second surface of the semi-permeable foil which is opposite to the first surface and the luminophore main body. Thus the luminophore can indeed in principle be reached by oxygen for quenching the excited radiation, but nevertheless protected from the effect of the measurement fluid wetting the contact surface. Although in principle the luminophore can be arranged at a surface of the luminophore main body, for technical production reasons it is preferable for the luminophore to be arranged in the foil or on the surface of the foil facing towards the luminophore main body.

An example of a material for producing an oxygen-permeable foil which can serve as a substrate for a luminophore layer, is polyvinylidene fluoride. It should, however, not be ruled out that the luminophore itself forms the first section of the contact surface and is directly wettable by the measurement fluid. Because of the disruptive effects of the measurement fluid on the luminophore through humidity and other contaminations carried along, however, this design is not preferred.

The surface of the oxygen-permeable foil forming the first section of the contact surface can be arranged flush with the second section of the contact surface. Alternatively, the surface of the oxygen-permeable foil forming the first section of the contact surface can be offset with respect to the second section of the contact surface in the direction of the depth extension, and thereby preferably also in the thickness direction of the luminophore main body, in magnitude approximately by the thickness of the foil. Additionally, the thickness of a luminophore layer carried by the oxygen-permeable foil can also contribute to the magnitude of the offset.

So that the first electromagnetic radiation can reach the luminophore through the luminophore main body in order to excite the luminophore and so that the excited second electromagnetic radiation can also reach the coupled radiation-sensitive sensor arrangement through the luminophore main body, the luminophore main body preferably exhibits for radiation in a first wavelength region from 450 to 480 nm and in a second wavelength region from 730 to 1100 nm a transmittivity each of at least 80%. Preferably of at least 90%, especially preferably of at least 95%. The aforementioned synthetic MABS also exhibits in the wavelength regions last mentioned the desirably high transmittivities. Preferably, therefore, the luminophore main body is made from MABS.

In principle it is conceivable to fabricate the main body of the temperature detection region and the luminophore main body separately and to join or assemble them into one main body of the radiation emission component. In an especially preferred embodiment, in contrast, the main body and the luminophore main body are configured integrally-monolithically as a radiation emission component main body. The main body and the luminophore main body each form a section of this radiation emission component main body. Preferably, the main body section and the luminophore main body section are arranged offset to one another in the reference direction.

Since both the luminophore main body and the base layer body can preferably be fabricated from the thermoplastic MABS and since further preferably the main body of the temperature detection region and the base layer body should be fabricated bonded integrally with one another, preferably the main body of the temperature detection region is also made from MABS. Consequently, in an especially preferred embodiment the radiation emission component main body and the base layer body bonded integrally with it are fabricated from MABS, more strongly preferably through injection molding or injection-compression molding.

The reference direction is preferably the longitudinal direction of the radiation emission component in a Cartesian coordinate system consisting of a longitudinal direction, width direction, and thickness direction. In the longitudinal direction, the radiation emission component exhibits its greatest dimension under the named Cartesian coordinate directions and consequently offers sufficient space for configuring the temperature detection region on the one hand and of the luminophore-containing region on the other next to each other.

The present invention concerns in an advantageous development a measuring cuvette with a radiation emission component, as described and developed above. The measuring cuvette exhibits a gas space with a receiving volume for receiving a measurement gas as the measurement fluid. The contact surface forms a wall of the gas space which bounds the receiving volume and is exposed towards the receiving volume. Furthermore, at least one section of the coupling surface is exposed towards the external environment of the measuring cuvette. Preferably, in order to facilitate the coupling of a radiation-sensitive sensor arrangement, the first and the second region of the coupling surface at the measuring cuvette face in identical directions. Preferably, at least the first region of the coupling surface is planar. Likewise, the second region of the coupling surface is preferably planar and especially preferably parallel to the first region of the coupling surface, although because of the recess it is arranged offset relative to the first region in the direction of the depth extension of the recess.

For the option of determining the carbon dioxide content in the measurement gas in addition to the option created through the radiation emission component of temperature-compensated detection of the oxygen content of the measurement gas, at least one boundary wall of the gas space exhibits a window transparent to radiation in the infrared wavelength region. Thus through infrared spectroscopy, due to the absorption behavior of carbon dioxide in the respiratory gas, its fraction in the latter can be determined. Preferably, the at least one boundary wall with the IR-transparent window is a different boundary wall than the wall formed by the radiation emission component or more specifically the contact surface. Especially preferably, the boundary wall exhibiting the IR-transparent window exhibits at its inner surface wetted by measurement gas a surface normal which encloses with the surface normal of the contact surface an angle of 80 to 100°, preferably of 90°. In an especially preferred manner, the measuring cuvette is configured for traversal of the gas space by infrared radiation for measuring the carbon dioxide content of the measurement gas, such that two boundary walls opposite to one another across the gas space each exhibit a window made from a material transparent to radiation in the infrared wavelength region.

In principle it is conceivable for the gas space to be filled with the measurement gas and the measurement gas to be detected metrologically after filling. The preferred application of the measuring cuvette, however, lies in the metrological detection of respiratory gas as the measurement gas, namely during its supply to the patient and/or its removal from the patient. Preferably, therefore, the gas space can be traversed by measurement gas along a throughflow direction, preferably along two opposite throughflow directions.

The measuring cuvette preferably forms part of a ventilation line, in order to supply a patient with inhalatory respiratory gas and remove exhalatory respiratory gas. The radiation emission component can therefore, in a preferred development of the present invention, lie in a throughflow direction between two connecting formations, where each of the connecting formations is configured for connecting a line conveying measurement gas. Entirely in principle, the measuring cuvette is not limited to applications in ventilation lines, although this is preferable.

The present invention concerns in a further advantageous embodiment a sensor device, comprising a measuring cuvette as described and developed above, and further comprising a radiation-sensitive sensor arrangement with a sensor housing. In the sensor housing there is accommodated at least:
A radiation source for radiating the first electromagnetic radiation,
A first radiation sensor for detecting the second electromagnetic radiation, and
At least one infrared sensor for detecting an infrared radiation in a wavelength region for which the base of the temperature detection region exhibits a transmittivity not exceeding 20%, preferably not exceeding 10%, especially preferably not exceeding 5%.

The sensor housing, preferably reusable after completion of a measurement task for subsequent measurement tasks, can be affixed detachably to a section of the measuring cuvette exhibiting the radiation emission component. Preferably, the sensor housing can be slid astride onto the section of the measuring cuvette exhibiting the radiation emission component and pulled off it. Likewise preferably, the sensor housing can be secured to the measuring cuvette in the coupled state by means of surmountable latching or detachable locking.

These and other objects, aspects, features and advantages of the invention will become apparent to those skilled in the art upon a reading of the Detailed Description of the invention set forth below taken together with the drawings which will be described in the next section.

### BRIEF DESCRIPTION OF THE DRAWINGS

The invention may take physical form in certain parts and arrangement of parts, a preferred embodiment of which will be described in detail and illustrated in the accompanying drawings which forms a part hereof and wherein:

FIG. 1 A longitudinal section view through a first embodiment of a radiation emission component of the present application,

FIG. 2 A perspective view of a second embodiment of a radiation emission component of the present application,

FIG. 3 A perspective view of a measuring cuvette with a radiation emission component of the present application,

FIG. 4 A frontal view of the measuring cuvette of FIG. 3 against the longitudinal direction L,

FIG. 5 A transverse sectional view of the measuring cuvette of FIGS. 3 and 4, where the offset sectional plane oriented orthogonally to the longitudinal direction L runs through the gas space of the measuring cuvette and through the recess in the temperature detection region of the radiation emission component,

FIG. 6 A perspective view of a radiation-sensitive sensor arrangement for detachable coupling with the measuring cuvette of FIGS. 3 to 5, and

FIG. 7 A bottom view of the radiation-sensitive sensor arrangement of FIG. 6.

### DESCRIPTION OF PREFERRED EMBODIMENTS

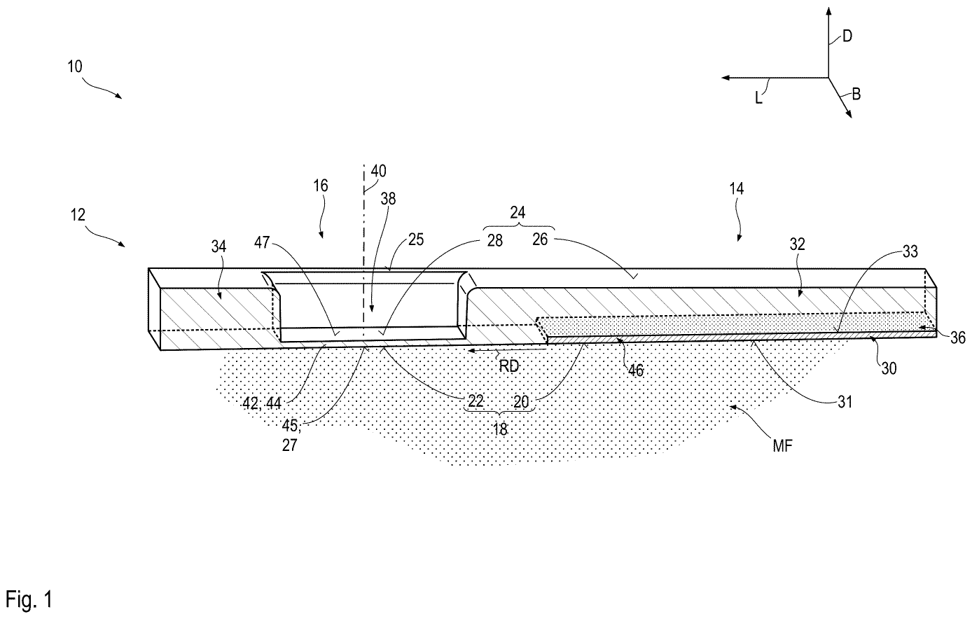

Referring now to the drawings wherein the showings are for the purpose of illustrating preferred and alternative embodiments of the invention only and not for the purpose of limiting the same, in FIG. 1, a first embodiment of a radiation emission component according to the invention is labelled generally by 10. The radiation emission component 10 comprises a radiation emission component main body 12 made from MABS, fabricated integrally through injection molding.

A Cartesian coordinate system consisting of pairwise mutually orthogonal coordinate directions: longitudinal direction L, width direction B, and thickness direction D, is specified in the drawings showing a radiation emission component in order to make clear the respective orientation of the radiation emission component in the relevant drawing.

The radiation emission component 10 exhibits a luminophore-containing region 14 and a temperature detection region 16, which are arranged offset relative to one another in the longitudinal direction L as a reference direction.

The radiation emission component 10 exhibits a contact surface 18 which during the operation of the radiation emission component 10 is wettable by a fluid to be measured, in particular gas. A section of the contact surface 18 lying in the luminophore-containing region 14 forms a first section 20 of the contact surface 18. A section of the contact surface 18 lying in the temperature detection region 16 forms a second section 22 of the contact surface 18.

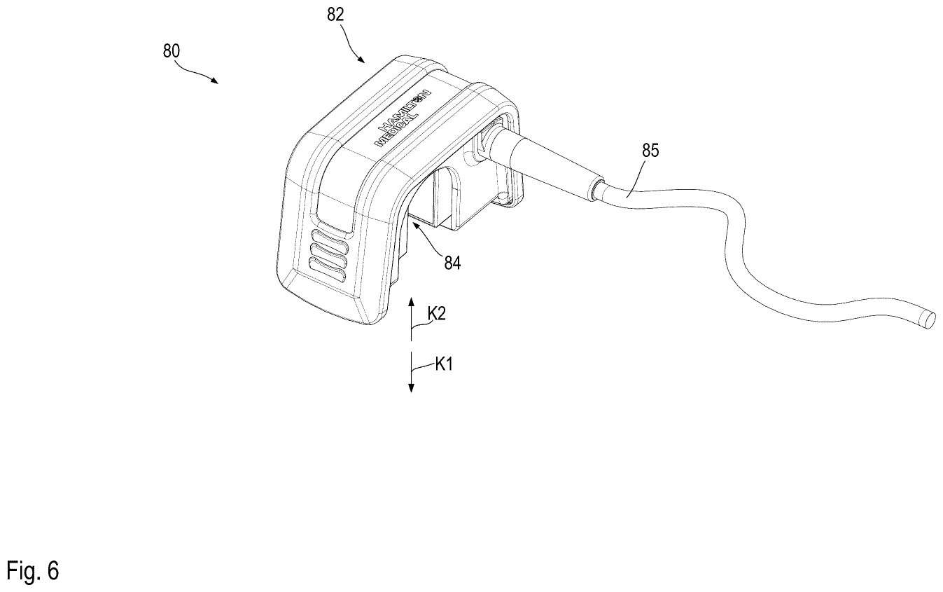

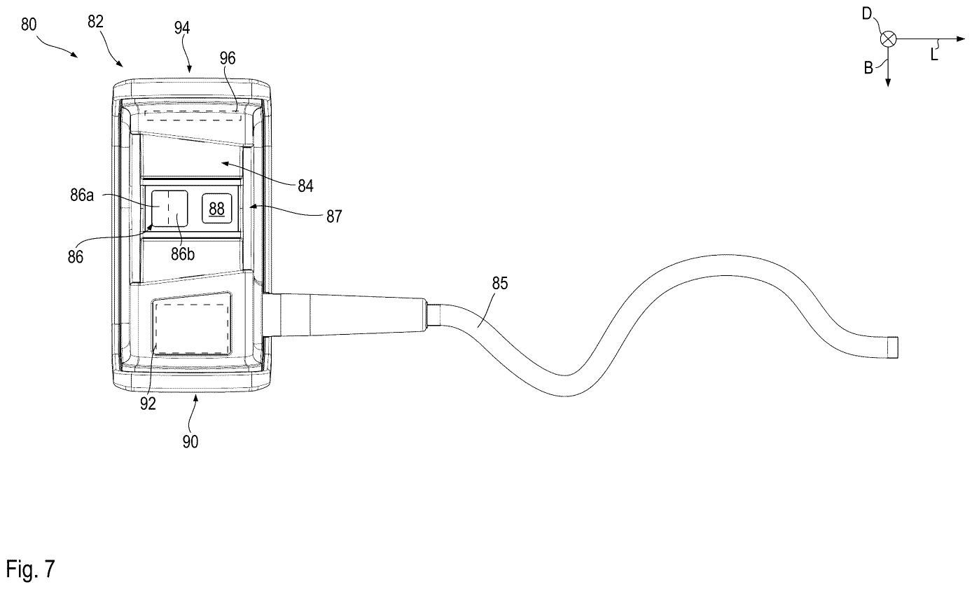

In the thickness direction D, arranged at a distance from the contact surface 18 or lying opposite it, as the case may be, the radiation emission component 10 exhibits a coupling surface 24, which serves for temporary detachable coupling with a radiation-sensitive sensor arrangement (see for example the radiation-sensitive sensor arrangement 80 in FIGS. 6 and 7) in order to transmit radiation information from the radiation emission component 10 to the radiation-sensitive sensor arrangement and to transmit excitation radiation, which in the descriptive introduction is designated first electromagnetic radiation, to the luminophore in the luminophore-containing region 14 in order to excite the latter to radiate a second electromagnetic radiation whose wavelength differs from that of the excitation radiation.

For the coupling surface 24 there apply analogously the statements made regarding the contact surface 18: A section of the coupling surface 24 lying in the luminophore-containing region 14 forms a first section 26 of the coupling surface 24 and a section of the coupling surface 24 lying in the temperature detection region 16 forms a second section 28 of the coupling surface 24.

The luminophore-containing region 14 is defined through the arrangement of the luminophore. An oxygen-permeable foil 30 made from polyvinylidene fluoride is bonded integrally in the luminophore-containing region 14 with a luminophore main body 32, for example through ultrasound welding. The luminophore main body 32 forms a section of the radiation emission component main body 12. The luminophore main body 32 is configured through injection molding integrally-monolithically with a main body 34 of the temperature detection region 16. The main body 34 likewise forms a section of the radiation emission component main body 12.

The foil 30 forms with the free surface 31 on its side facing away from the luminophore main body 32 the first section 20 of the contact surface 18 and carries on its opposite surface 33 a thin layer 36 indicated by tight dots with the luminophore. The luminophore is consequently arranged between the luminophore main body 32 and the foil 30 and protected against outside effects. The foil 30 made from polyvinylidene fluoride is oxygen-permeable, such that oxygen molecules can arrive via the contact surface 18 or to be precise via the first section 20 of same at the luminophore in the layer 36 in the luminophore-containing region 14 and can affect its radiation behavior through quenching in a manner known per se. The quantity of oxygen molecules arriving at the luminophore per unit of time is proportional to the partial oxygen pressure in the measurement fluid MF wetting the contact surface 18 or to be precise the first section 20 of same. The measurement fluid MF is symbolized in the depiction as a cloud of dots with a lower dot density as that of the layer 36.

The temperature detection region 16 is characterized by a blind hole-like recess 38. The recess 38 extends starting from a free surface 25 of the radiation emission component main body 12 or to be precise of the main body 34 which is opposite the contact surface 18 in the thickness direction D along an axis 40 of the depth extension towards the contact surface 18. In the depicted embodiment example, the surface 25 forms with the second section 26 of the coupling surface 24 a common planar surface.

The recess 38 is covered by a base 42 which in the depicted embodiment example is formed by a base layer body 44 configured in the injection molding integrally with the main body 34. The consequently exclusively from MABS formed base layer body 44 forms with its free surface 45 facing away from the recess 38 the second section of the contact surface 18 and forms with its opposite free surface 47 facing towards the recess 38 the second section of the coupling surface 24.

The base layer body 44, due to its formation out of MABS, is quasi-opaque or to be precise exhibits a transmittivity of less than 5% in the wavelength region from 5,500 to 14,000 nm relevant for the infrared temperature detection. The base layer body 44 exhibits in the depicted embodiment example a thickness of approximately 200 μm to 240 μm.

Through its integrally-monolithic configuration together with the main body 34 and/or with the radiation emission component main body 12 as the case may be, it is highly firmly arranged at the main body 34 and bonded with the latter. Detachment of the base layer body 44 from the main body 34 is conceivable in the depicted embodiment example only through willful destruction of the temperature detection region 16 of the radiation emission component 10. This requires a considerable exertion of force, which significantly exceeds the forces occurring during the proper operation. At the same time, the base layer body 44 together with the main body 34 and the radiation emission component main body 12 can very advantageously be fabricated in a single working step.

The foil 30 exhibits a similar thickness to the base layer body 44 and exhibits as a synthetic a similar heat capacity and a similar heat conductance, such that when the contact surface 18 is wetted with measurement fluid, the luminophore layer 36 on the surface 33 of the foil 30 opposite the first section 20 of the contact surface 18 and the second section 28 of the coupling surface 24 exhibit approximately the same temperature. This applies in an advantageous manner both in the quasi stationary state and when regarded in a transient state, for instance if the temperature of the measurement fluid at the contact surface 18 changes and the measurement fluid transfers this temperature change to the contact surface 18.

As can be discerned in FIG. 1, the first section 20 and the second section 22 of the contact surface 18 are arranged offset to one another not only in the longitudinal direction L as a possible reference direction, but also in a reference direction RD parallel to the contact surface 18, where in the depicted embodiment example the reference direction RD exhibits its greatest dimension component in the longitudinal direction L.

In the embodiment example of FIG. 1, there is configured in the luminophore main body 32 a recess 46 in which the foil 30 is preferably so accommodated that the first section 20 and the second section 22 of the contact surface 18 are arranged flush and lie in a common plane. The surface 27 of the radiation emission main body 12 facing towards the measurement fluid MF exhibits a jump in the transition to the recess 46.

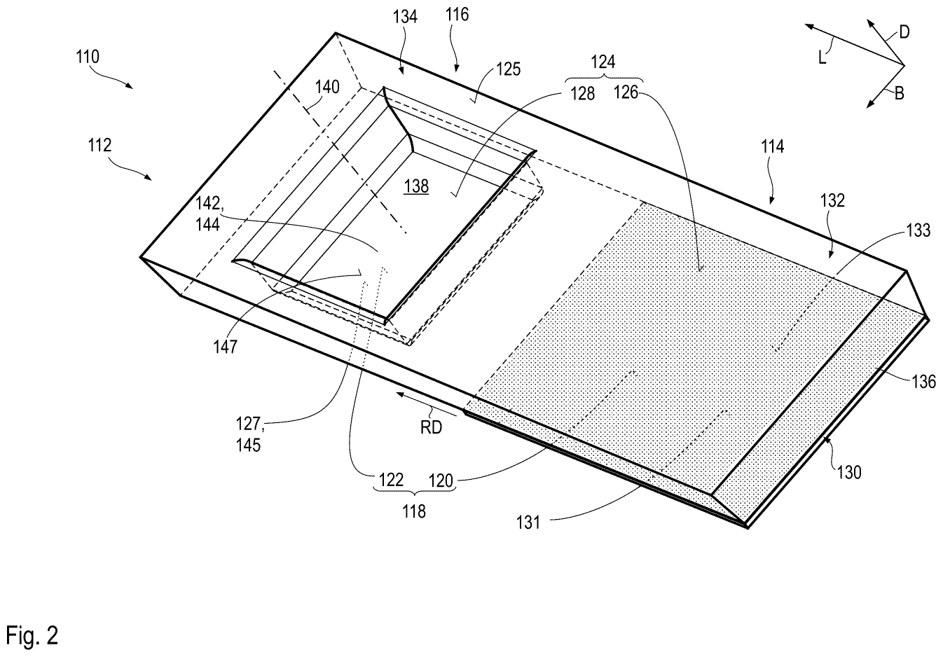

In FIG. 2 there is depicted in a perspective view a second embodiment of a radiation emission component according to the invention. Identical and functionally identical components and component sections as in the first embodiment depicted in FIG. 1 are labelled in FIG. 2 with the same reference marks, but increased numerically by 100.

The second embodiment shown in FIG. 2 is described below only in so far as it differs from the first embodiment of FIG. 1, to whose description otherwise reference is also expressly made to elucidate the second embodiment.

In the second embodiment, the foil 130 with the luminophore layer 136 is affixed to the surface 127 which is configured as planar, jump-and kink-free of the radiation emission component main body 112 through ultrasound welding. The surface 127 in the region of the temperature detection region 116 forms part of the second section 122 of the contact surface 118. The first section 120 of the contact surface 118 is offset with respect to the second section 122 by the thickness of the foil 130 and by the effectively negligible thickness of the luminophore layer 136 in the thickness direction D, that is, in direction of the axis 140 of the depth extension of the recess 138.

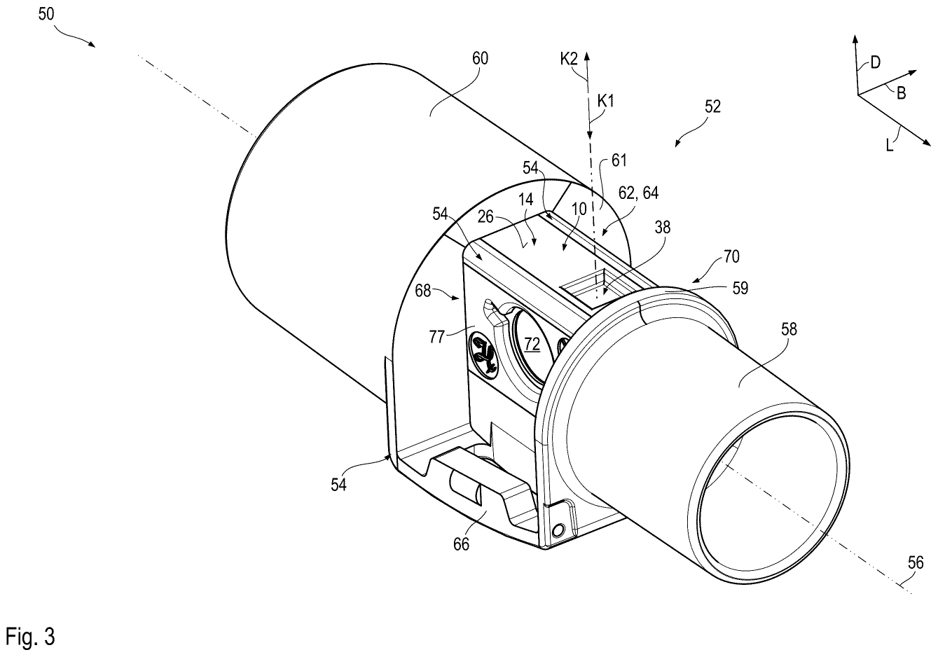

In FIG. 3, a measuring cuvette 50 with the radiation emission component 10 is depicted in perspective. Instead of the radiation emission component 10, which is chosen merely as an example, the measuring cuvette 50 could also exhibit the radiation emission component 110.

The measuring cuvette comprises a cuvette housing 52, of which the radiation emission component 10 forms a part. The rest of the cuvette housing 54 without the radiation emission component 10 is likewise predominantly by weight or completely made from MABS and is preferably fabricated in the injection-molding method. The IR-transparent windows 72 and 74 and their frame parts 77 and 78 too, are preferably fabricated in the injection-molding method. Because of the identity of the materials, the part of the rest of the cuvette housing 54 produced by injection molding can be simply injected onto the radiation emission component 10 without further joining measures to form the cuvette housing 52.

The cuvette housing 52 can have flow through it bidirectionally along the throughflow path 56, which in the depicted example is preferably configured as a straight line. The cuvette housing 52 is configured for use in a ventilation line. It exhibits a distal connecting formation 58 and a proximal connecting formation 60 onto each of which further sections of the ventilation line can be connected. In the envisaged installation position, a ventilation line section connected to the proximal connecting formation 60 leads to the ventilated patient and a ventilation line section connected to the distal connecting formation 58 leads to the ventilation device.

Between the two connecting formations 58 and 60 along the throughflow path 56 there is configured a coupling section 62 with a coupling formation 64 which is detachably couplable with the radiation-sensitive sensor arrangement 80 shown in FIGS. 6 and 7. The radiation-sensitive sensor arrangement 80 can be slid along the arrow K1 astride onto the coupling section 62 and pulled off the coupling section 62 in the opposite direction along the arrow K2. A coupling structure 66 tapering from the proximal to the distal end on the end side, bottom side in the direction of the arrow K1 of the coupling section 62 prevents coupling of a sensor arrangement 80 oriented incorrectly and in addition allows detachable latching of the sensor arrangement 80 at the coupling section 62.

The distal connecting formation 58 ends at a wall 59 oriented essentially orthogonally to the throughflow path 56, which separates the connecting formation 58 from the coupling section 62. At its proximal longitudinal end, the coupling section 62 ends in a wall 61, starting from which the proximal connecting formation 60 extends away from the coupling section 62.

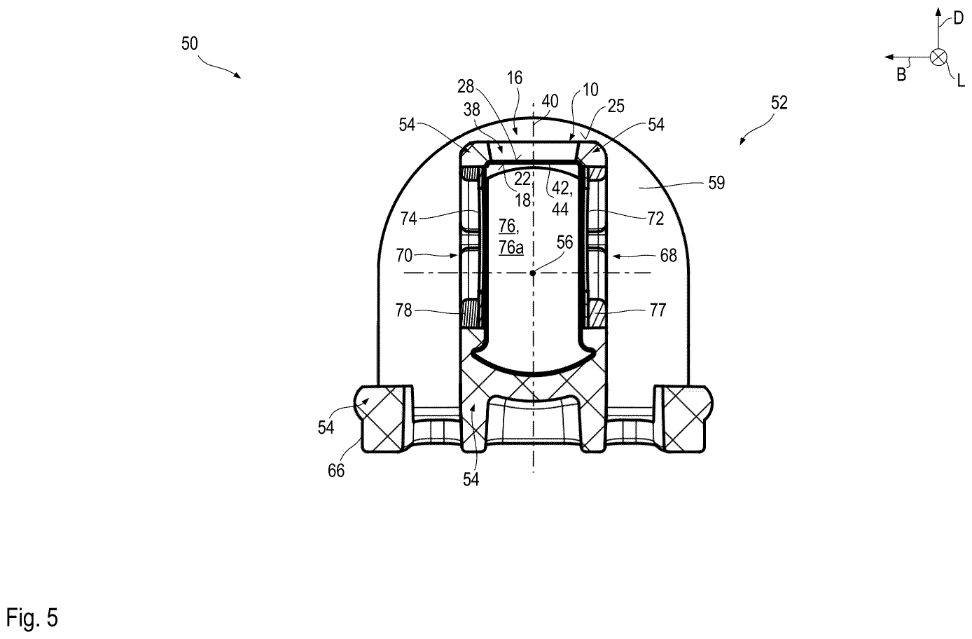

In the depicted embodiment, the radiation emission component 10 forms a ceiling section of the coupling section 62. Side windows 68 and 70 (see also FIG. 5) oriented transversely, preferably orthogonally, to the ceiling section each exhibits a side window 72 and 74 respectively transparent to infrared radiation, in order to be able to pass infrared radiation through a traversable gas space 76 lying in the interior of the coupling section 62. Thereby, an infrared spectroscopical detection of the carbon dioxide content or in principle of another carbon dioxide-absorbing gas of the measurement gas flowing in the gas space 76 is possible.

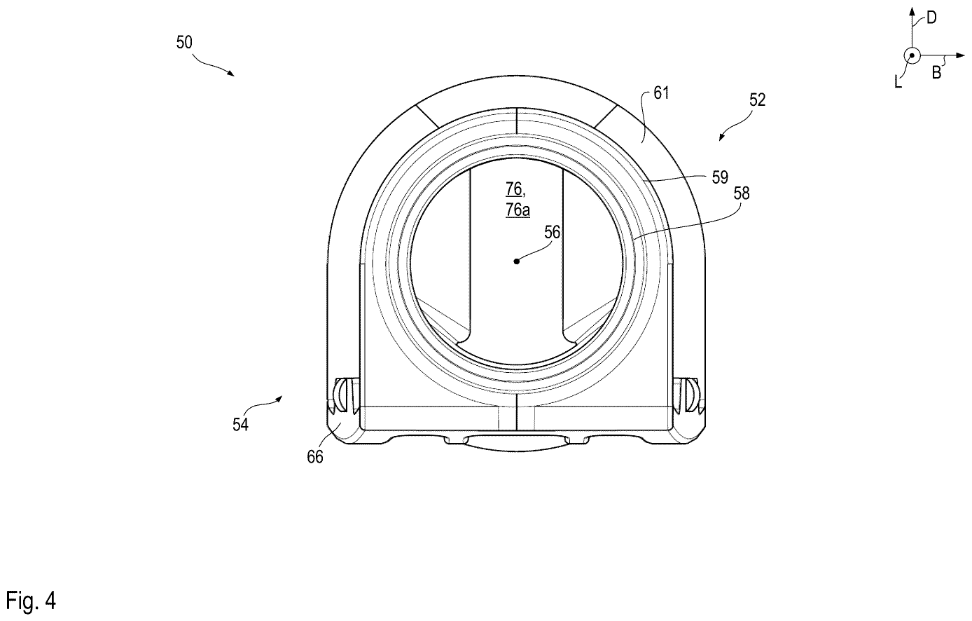

The observer of FIG. 4 looks along the throughflow path 56 from the distal longitudinal end of the measuring cuvette 50 into the gas space 76 of same. The gas space 76 exhibits a receiving volume 76a into which the measurement gas MF can be received, also as a flowing measurement gas MF.

FIG. 5 shows a cross-section through the measuring cuvette 50 along an offset sectional plane, with a first planar section which extends oriented orthogonally to the throughflow path 56 in the region of the radiation emission component 10 through its recess 38, and with a second planar section parallel to the first one, which extends starting from the second section 22 of the contact surface through the midpoints of the circular side windows 72 and 74 respectively. The observer of FIG. 5 looks starting from the sectional area in the direction towards the distal connecting formation 58. The side windows 72 and 74 are connected with frames 77 and 78 respectively, which are connected integrally with the rest of the cuvette housing 54 through injection molding. The contact surface 18 of the radiation emission component 10 forms a wall of the gas space 76, which bounds its receiving volume 76a above.

In FIGS. 6 and 7 there is depicted a radiation-sensitive sensor arrangement 80 couplable detachably with the measuring cuvette 50, more accurately with its coupling section 62. FIG. 6 shows a perspective view of the sensor arrangement 80, FIG. 7 a bottom view of it. The sensor arrangement 80 comprises a sensor housing 82 with a through-opening 84 surrounded on three sides by the sensor housing 82, in which the coupling formation 62 of the measuring cuvette 50 is situated in the coupled with the measuring cuvette 50 state of the sensor arrangement 80.

On the side of the sensor housing 82 which in the coupled with the measuring cuvette 50 state faces towards the distal connecting formation 58 of the measuring cuvette 50, there is connected with the sensor housing 82 an electric cable 85 which both supplies electric functional units accommodated in the sensor housing 82 with electric current and delivers control signals to the functional units and transmits detection signals from the functional units to a superordinate control device or evaluation device.

In the bottom view there is discernible as such a functional unit a sensor unit 86 which cooperates with the luminophore-containing region 14 of the radiation emission component 10 of the measuring cuvette 50. This sensor unit 86 comprises a radiation source 86a which is configured to emit a first electromagnetic radiation as excitation radiation. The sensor unit 86 further comprises a radiation sensor 86b which is configured to detect an excited second electromagnetic radiation which is emitted by the luminophore-containing region 14 due to this excitation.

There is further discernible in the bottom view as a further such functional unit an infrared sensor 88 which is configured to detect infrared radiation emitted by the second section 28 of the coupling surface 24.

The sensor unit 86 and the infrared sensor are accommodated in a housing section 87 bridging the through-opening and in the coupled with the measuring cuvette 50 state lie directly opposite the coupling surface 24 of the radiation emission component 10.

In the sensor housing region 90 of the sensor housing 82 located on a side of the through-opening 84 in which the electric cable 85 terminates, there is accommodated as a third such functional unit an infrared radiation source 92 which emits infrared radiation over the width of the through-opening 84 to the sensor housing region 94 located on the other side of the through-opening 84, opposite to the sensor housing region 90, where a second infrared sensor 96 is accommodated as a fourth such functional unit. The second infrared sensor 96 detects the infrared radiation emitted by the infrared radiation source 92 after passing through the two side windows 72 and 74 and through the gas space 76. Thereby is made possible infrared spectroscopic quantitative detection of the carbon dioxide content of a gas flowing through the gas space 76.

While considerable emphasis has been placed on the preferred embodiments of the invention illustrated and described herein, it will be appreciated that other embodiments, and equivalences thereof, can be made and that many changes can be made in the preferred embodiments without departing from the principles of the invention. Furthermore, the embodiments described above can be combined to form yet other embodiments of the invention of this application. Accordingly, it is to be distinctly understood that the foregoing descriptive matter is to be interpreted merely as illustrative of the invention and not as a limitation.

1-15. (canceled)

16. A radiation emission component for temperature-compensated optical detection of an oxygen content of a fluid, where the radiation emission component comprises a contact surface wettable by the fluid and a coupling surface different from the contact surface for coupling with a radiation-sensitive sensor arrangement,
where the radiation emission component exhibits a luminophore-containing region, where an excited radiation behavior of the luminophore in the luminophore-containing region depends on a partial oxygen pressure in a fluid wetting the contact surface, and where the luminophore can be reached by oxygen in a first section of the contact surface,
where the radiation emission component exhibits an infrared radiation-emitting temperature detection region, where the temperature detection region is arranged offset relative to the luminophore-containing region in a reference direction along the contact surface, and
where the radiation emission component exhibits in the section in which the temperature detection region is arranged a main body with a recess extending towards the contact surface, where the recess is covered by a base comprising a base layer body, where a surface of the base layer body facing away from the recess forms a second section of the contact surface different from the first section,
where the main body and the base layer body each comprise a synthetic material, where the main body and the base layer body are connected integrally with one another by means of their respective synthetic materials.

17. The radiation emission component according to claim 16, wherein the synthetic material of the main body and the synthetic material of the base layer body are fused with one another regionally or wherein the main body and the base layer body are formed integrally-monolithically from the same synthetic material.

18. The radiation emission component according to claim 17, wherein the base layer body is made completely from the synthetic material.

19. The radiation emission component according to claim 16, wherein the base layer body is made completely from the synthetic material.

20. The radiation emission component according to claim 16, wherein the base layer body is configured as thinner than the main body surrounding the recess.

21. The radiation emission component according to claim 16, wherein the synthetic material of the base layer body exhibits for infrared radiation in a wavelength region from 5500 nm to 14000 nm a transmittivity not exceeding 20%.

22. The radiation emission component according to claim 21, wherein the synthetic material of the base layer body exhibits for infrared radiation in the wavelength region a transmittivity not exceeding 5%, where the synthetic material of the base layer body forms the surface of the base facing towards the recess.

23. The radiation emission component according to claim 22, wherein the base layer body carries a coating on its surface facing away from the contact surface and/or on its surface facing towards the contact surface, where the base exhibits in the direction towards the recess an absorptance of at least 95% and/or an emissivity of at least 95% in the wavelength region from 5500 nm to 14000 nm.

24. The radiation emission component according to claim 16, wherein the base layer body carries a coating on its surface facing away from the contact surface and/or on its surface facing towards the contact surface, where the base exhibits in the direction towards the recess an absorptance of at least 95% and/or an emissivity of at least 95% in the wavelength region from 5500 nm to 14000 nm.

25. The radiation emission component according to claim 16, wherein the radiation emission component exhibits a luminophore main body, where the luminophore main body carries an oxygen [symbol] - permeable foil, where a first surface of the foil forms the first section of the contact surface of the radiation emission component.

26. The radiation emission component according to claim 25, wherein the luminophore main body exhibits for radiation in a first wavelength region from 450 to 480 nm and in a second wavelength region from 730 to 1100 nm a transmittivity each of at least 80%.

27. The radiation emission component according to claim 26, wherein the main body and the luminophore main body are configured integrally-monolithically as a radiation emission component main body, of which the main body and the luminophore main body each form a section.

28. The radiation emission component according to claim 27, wherein in a Cartesian coordinate system consisting of a longitudinal direction, width direction, and thickness direction, the reference direction is the longitudinal direction of the radiation emission component in which the radiation emission component exhibits its greatest dimension under the named Cartesian coordinate directions.

29. A measuring cuvette with a radiation emission component according to claim 16, where the measuring cuvette exhibits a gas space with a receiving volume for receiving a measurement gas, where the contact surface forms a wall of the gas space which bounds the receiving volume and is exposed towards the receiving volume and where at least one section of the coupling surface is exposed towards the external environment of the measuring cuvette.

30. The measuring cuvette according to claim 29, wherein at least one boundary wall of the gas space exhibits a window transparent to radiation in the infrared wavelength region.

31. The measuring cuvette according to claim 30, wherein the gas space can be traversed by measurement gas along a throughflow direction, where the radiation emission component lies in the throughflow direction between two connecting formations, where each of the connecting formations is configured for connecting a line conveying measurement gas.

32. The measuring cuvette according to claim 29, wherein the gas space can be traversed by measurement gas along a throughflow direction, where the radiation emission component lies in the throughflow direction between two connecting formations, where each of the connecting formations is configured for connecting a line conveying measurement gas.

33. A sensor device, comprising a measuring cuvette according to claim 29 and further comprising a radiation-sensitive sensor arrangement with a sensor housing, in which there is accommodated:
a radiation source for radiating the first electromagnetic radiation,
a first radiation sensor for detecting the second electromagnetic radiation, and
at least one infrared sensor for detecting an infrared radiation in a wavelength region for which the base of the temperature detection region exhibits transmittivity not exceeding 20%,
where the sensor housing can be affixed detachably to a section of the measuring cuvette exhibiting the radiation emission component.

34. The sensor device according to claim 33, wherein at least one boundary wall of the gas space exhibits a window transparent to radiation in the infrared wavelength region.

35. The sensor device according to claim 34, wherein the gas space can be traversed by measurement gas along a throughflow direction, where the radiation emission component lies in the throughflow direction between two connecting formations, where each of the connecting formations is configured for connecting a line conveying measurement gas.
